(19) United States
(12) Patent Application Publication
(10) Pub. No.: US 2019/0275270 A1
(43) Pub. Date: Sep. 12, 2019

(54) SYSTEM AND METHOD FOR ARRESTING DEBILITATING MIGRAINE EVENTS

(71) Applicant: Richard Postrel, Miami Beach, FL (US)

(72) Inventor: Richard Postrel, Miami Beach, FL (US)

(21) Appl. No.: 15/913,983

(22) Filed: Mar. 7, 2018

(51) Int. Cl. A61M 15/00; A61K 9/00; A61M 11/08; A61K 31/352

(52) U.S. Cl. CPC A61M 15/0068; A61K 9/0043; A61K 38/04; A61K 31/352; A61M 11/08

(57) ABSTRACT

A system and method for treating acute migraine events utilizing multiple sensory/receptive channels (olfactory, audio, visual and other sensory receptors) to both treat and optimize delivery to the afflicted sites in the brain. One of the primary migraine treatment methods includes a composition delivered through the olfactory system. Absorption through the nasal membranes is rapid and avoids delays and dilution inherent in methods using the mouth and gastro-intestinal (GI) processing. Effective, but less preferred methods that avoid GI delays and dilution include delivery as eyedrops or as an intravenous infusion. The effective composition comprises a caffeinic substance to bind and antagonize adenosine receptors in the brain. This immediately blocks continued action of adenosine to dilate the afflicted vessels and inflict pain. The composition also incorporates cannabinolic substances which work simultaneously or in parallel using the body's natural endocannabinoid pathways to rebalance and control blood flow to minimize swelling and pain. Additional features may incorporate additional active components. One available feature may incorporate a recipient/patient control modulus where the patient adjusts treatment using a feedback device such as a stimulation button to increase or adjust delivery. Another additional feature comprises one or more essential oils. Although some essential oil compositions may themselves include one or more compounds that contribute to the desired cannabinolic effects, adding essential oils even those lacking an inherent cannabinolic effect can contribute to effective treatment. Fragrant inclusions in an essential oil additive may provide a calming stimulation to the olfactory system that supports reduced tension and relaxation and thereby removes a tension induced exacerbation of migraine progression.

### BACKGROUND

Migraine is a common and still inadequately treated neurological disorder. With or without aura migraines constitute the most common cause of severe, recurring headache. But headache is only one of the ways the disease manifests. Migraine can also include visual disturbances, alterations in consciousness, photophobia, and/or phonophobia. Migraines can be truly debilitating; the pain often interferes with a person's ability to live a normal productive life. Lingering symptoms can force sufferers to abandon everyday activities for several days. Even in symptom-free period, sufferers often live in fear of the next attack. It has been estimated that as many as 1 in 7 or 8 adults have migraine events.

### SUMMARY OF THE INVENTION

This invention recognizes the severity of migraine events and teaches a rapid effective method of treating or managing the migraine at its inception. Rapid treatment is essential. Accordingly, delivery through the olfactory system is recognized as a preferred method of delivery. Since migraines arise from hyperexcitability of adenosine receptors, antagonizing these receptors is a first target for immediate relief. The endogenous cannabinoid receptor systems are an important calming and secondary target. The delivered medicament may include an essential oil or similar compound which may exert effects though one of these two mechanisms of action, but often will have significant secondary, side or independent beneficial activities.

Preventative treatment is often utilized when migraines occur frequently, e.g., more frequently than once each week, or when migraines are less frequent, but symptoms are severely debilitating. The goal of preventative or prophylactic treatment is to lessen the frequency and severity of the migraine attacks. Medication to prevent a migraine can be taken daily. Preventive treatment medications include but are not limited to: a) medications used to treat high blood pressure, e.g.: beta-blockers (propranolol, timolol, metoprolol), calcium channel blockers (verapamil), etc.; b) antidepressants: amitriptyline, nortriptyline (Aventyl, Pamelor), etc.; c) antiseizure medications, e.g.: gabapentin (Neurontin), topiramate (Topamax), valproic acid (Depakote), etc.; d) paralytics, e.g.: Botox, etc.; etc.

Understanding the molecular mechanisms that precede and give rise to a migraine attack is key to developing new therapeutic strategies. Advances towards this goal have recently been made through genome-wide association studies, which have identified new genetic components of migraine that highlight vascular etiologies and underline the polygenic nature of this disorder.

Nontraditional supplement treatments for migraine prevention include but are not limited to: butterbur, coenzyme Q10 (CoQ10), feverfew, etc., with mixed results.

But in the thralls of a migraine attack or an imminent migraine attack (e.g., during or shortly after aura), abortive treatment is standard protocol. Abortive medications are intended to stop as a migraine the victim senses one coming on or soon after it has begun. Abortive medications may be administered or taken by injection, mouth, skin patch, buccal drops, nasal spray, etc. Non-oral forms of medication are especially useful for people who have nausea or vomiting related to their migraine, and they work quickly.

In view of the desire for speed, abortive interventions for migraine activity preferably consider avoiding delays wherever possible. For example, oral dosing of medications generally incurs a delay of about 20 minutes as the medicament travels through the GI track from which it is absorbed into the blood coursing through the circulatory system where the medicament is diluted, bound by proteins or cells and delivered throughout the entire body. Significant amounts of substance are wasted, and the wide distribution raises additional questions regarding undesired side effects. Local injection is an option for some treatments, but such injection is not possible directly into the brain. Sound or electromagnetic harmonics may be put to essentially instantaneous and immediate interactions with areas upon which they are focused. Perhaps a harmonic sound of about 70 Hz, perhaps cycling between about 50 Hz and 90 Hz, or cycling in response to user input or internal responses that might be detected by electromagnetic, temperature or other biomarker sensor is used in practice to optimize the harmonic treatment. As mentioned above, second stage cycling may involve cycling through frequencies. Cycling may also involve a cyclic overlay of volume of the main harmonic sound reducing and/or increasing the volume over a static frequency or modulating volume as a function of a cycling frequency. One promising pattern involves a base harmonic frequency around which a secondary frequency fluctuates. Harmony between these two or more frequencies varies as a function of the different ratios. For example, frequencies involving both a 4^{th} and 5^{th} harmonic interplaying off each other may show promise in calming migraine excitabilities. Given the complex three dimensional geometries of the brain and skull, and the dependence of the speed of a sound wave as a function of bio-density (the principle allowing ultrasonic imaging), the cycling frequencies will find different harmonic amplifications in changing three dimensional vectors as the brain tissue presents with different path lengths for the sound in the three dimensional space. As different changing frequencies deliver increasing and decreasing relief as a function of time and frequency user feedback can be used to optimize pain relieving results.

One embodiment features a simple signaling system such as a button or joystick like device through which the user signifies a perception of positive and or negative results of treatment. A simple system may merely focus frequencies and/or amplitudes towards the stimuli indicated as positive. A more robust system employs a system of artificial intelligence to gather data and better anticipate positive pain-relieving outputs for optimized or maximum relief. As an alternative or in addition to the user input interface, a system of sensors can be used allowing additional factors to build a more complex algorithm for managing migraine treatment and hopefully to discern indicative factors that lead to debilitating migraines and provide one or more signal(s) to migraine avoidance. Any type of bio-sensor may be used; preferably the biosensor is non-invasive, but if invasive, preferably installed for extended, possibly permanent, use. For example, a sensor system may monitor harmonic strength through vibration of the skull at various points. Precise changes in harmonic amplification, especially of a ratio of harmonic frequencies can indicate blood flow and other tissue adaptations for either positive or negative migraine management. A separate frequency may be used for imaging—with care to avoid undesired heating which may support further arteriole delivery of blood to dilated vessels. Ultrasonic stimulation might be used to divert blood from areas causing pain to less problematic locations. In preferred embodiments an artificial intelligence system is using stimuli to modulate blood flow, temperature or other migraine significant bio-feature as needed locally. Algorithms can be stored for individual patients for each session and used by the AI system to improve each subsequent session. With careful maintenance of privacy levels desired by each patient interpatient data analysis can be used to construct algorithms on an individual basis to optimize management of each migraine event.

In some embodiments the cranium is surrounded by or enclosed in a cap-like accessory. Especially for patients with light induced migraines the cap includes a face shield or blinders. It may incorporate a sophisticated data collection and analysis capability. It may include facility to deliver one or a plurality of chemical treatments through the mouth and/or nose. Sophisticated devices will respond to patient's selections by delivering an individual compound or a selected ratio of treatment compounds based on an individual response to user input and/or instructions provided by the AI system. A manual override to allow the recipient or another individual to terminate, restart, refocus or otherwise ignore algorithm supplied instruction is a desired feature.

Such cap may include another immediate therapy in the embodiment of electromagnetic radiation (light). While some migraine sufferers prefer or require absolute darkness, others benefit from calming visual environments. Specific colors may increase or decrease brain harmonics to modulate migraine event activities. This photo therapy may be included in a cap device or may be used in embodiments that do not include a cap.

Yet another therapy bypassing GI absorption is smell. Aromatherapy, e.g., breathing an essential oil that usually is extracted to produce a soothing aroma can be practiced using heated oils in the room, oils spread on lips, forehead, buccally, etc. The odor portion of aroma therapy is through the nose, our nasal allowing rapid delivery to the brain. Such features may be incorporated in a cap dispenser or may be used independently.

A preferred embodiment of this invention uses the rapid delivery system through the nose to minimize delays in responding to a migraine event. Nasal delivery (in addition to being a relatively non-invasive delivery method) for many substances may overcome issues related to poor bioavailability, slow absorption, drug degradation, may avoid adverse events in the GI tract and can bypass portal vein conducted metabolism in the liver. The narrow nasal passages comprising complex convoluted nasal geometry with absorbent surfaces provide efficient filtration, temperature and moisturizing conditioning of inspired air. These features are exploited to optimize gas exchange and absorption of inhaled substances that will contact the nasal linings during normal inspiration and expiration. As prepared for this invention, controlled delivery is preferred. Solid or atomized particles are preferably >˜9μ to insure contact with nasal membranes and avoid delivery to the lungs where the drug may be ineffective or may have unwanted effect. A drying agent to decrease mucus content may be incorporated in the delivered composition to improve speed of absorption and to counteract possible induction of mucus secretion by the carriers or active ingredients. A composition may comprise one or a plurality of compounds and may comprise metabolites or spontaneous isomers of an introduced compound.

Antimicrobial compounds like a benzalkonium salt may be added for a preservative effect. Essential oils may incorporate inherent antimicrobial, antioxidant and other preservative functions. Dispensers may include hand held devices that propel dry particles, liquid enveloped particles, liquid phase particles, gaseous active ingredients or propellants, etc., into the nasal airways. Other means are not excluded. For example, a static mask into which a recipient situates the nose or face may be used; a wearable mask may be used; a tent surrounding the body or a part thereof may be used to deliver desired treatments.

### BRIEF DESCRIPTION OF THE DRAWING

FIG. 1 is a block diagram of a preferred embodiment of the present invention.

FIG. 2 is a flowchart of the operation of the preferred embodiment of FIG. 1.

### DETAILED DESCRIPTION OF THE INVENTION

Conventional, but inadequate, abortive treatments include especially triptans that preferentially or specifically target serotonin pathways. Triptans are used to treat the headache only, and rarely relieve pain from back problems, arthritis, menstruation, inflammation, etc. Commonly used tryptans include but are not limited to: almotriptan (Axert), eletriptan (Relpax), frovatriptan (Frova), naratriptan (Amerge), rizatriptan (Maxalt), sumatriptan (Alsuma, Imitrex, Sumavel), zolmitriptan (Zomig), and the like. These medicaments have similar modes of action and chemical structures. Triptans act as selective 5-hydroxytryptamine (5-HT) receptor agonists with high affinity for 5-HT_{1B}, a vasoconstriction agent, and 5-HT_{1D}, an inhibitor of vasoactive peptide release receptors. Triptans are believed to relieve migraine pain by reducing neurogenic inflammation, lessening vasoconstriction of meningeal vessels, and modulating second-order neurons. Sumatriptan and rizatriptan are metabolized by monoamine oxidase A (MAO-A), eletriptan, naratriptan, and frovatriptan are metabolized by CYP enzymes. Zolmitriptan is metabolized by CYP_{1A2} with its active metabolite (183C91) metabolized by MAO-A; almotriptan, is metabolized by MAO-A, CYP enzymes and flavin monooxygenase 3. Metabolic fates and biologic half-lives of these and any chemicals metabolized in our bodies will be impacted by chemicals that inhibit expression or activity of the metabolic enzymes or ligands competing with the chemical of interest.

Sometimes drugs with more general applications such as: Acetaminophen-isometheptene-dichloralphenazone (Midrin), Dihydroergotamine (D.H.E. 45 Injection, Migranal Nasal Spray), Ergotamine tartrate (Cafergot) may be used for treatment. These also include many over-the-counter medications (in some markets) such as Advil-Migraine (containing ibuprofen), Excedrin-Migraine (containing aspirin, acetaminophen, caffeine), and Motrin-Migraine Pain (containing ibuprofen), etc.

In addition, several treatments involving one or more of the following drugs are sometimes used for nausea related to migraine headaches; products such as these may be taken after or during primary migraine treatment: Metoclopramide (Reglan), Prochlorperazine (Compro,), Chlorpromazine (Thorazine), Droperidol (Inapsine), etc.

Some well-known drugs are used for headache pain but are not specific for migraines. These generally include analgesics, narcotics, and barbiturates. Since many of these can be habit forming or addictive, they are less desirable than specific headache drugs like those mentioned above. These drugs are therefore most often used as a “backup” for the occasions when a specific drug does not work.

Understanding the molecular mechanisms that precede and give rise to a migraine attack is a key to developing effective therapeutic strategies. Advances towards this goal have recently been made through genome-wide association studies, which have identified several genetic components of migraine that implicate vascular etiologies and emphasize the polygenic nature of this disorder. One commonly noted vascular event relates to adenosine binding with its receptor to extensively open the affected vasculature. Caffeine, a common drug substance found in foods such as chocolate and drinks such as coffee and many soda beverages, also binds adenosine receptors and can interfere with adenosine binding and subsequent activities.

Caffeine, itself, is an addictive substance because the molecule fits precisely into a binding site on the brain's adenosine receptors. When these receptors are blocked with caffeine molecules, dopamine (the feel-good chemical) works more efficiently; the excess adenosine signals the adrenal glands to release adrenaline, which further perpetuates the feeling of alertness. When a person misses or decides to quit their usual caffeine dosage, the brain is then overwhelmed by normal levels of adenosine now free to bind the receptors vacated by caffeine—so that dopamine levels drop drastically and cause the brain's chemistry to go out of balance.

Caffeine is rapidly and completely absorbed in humans, with at least 99 percent being absorbed through the GI track within 45 minutes following ingestion, and even more rapid absorption when taken through other means, including, but not limited to: buccally, as a nebulized mist or spray, nasal jet spray, as small particulates, by disbursed evaporative resultant powder, etc. These methods of administration avoid the time necessary to traverse portions of the GI track where absorption may compete with absorption of other foods and the uptake or diffusion times since intestinal walls tend to be much thicker than other absorptive membranes. Predominant metabolism of caffeine and other theophyllines is through the hepatic enzyme CYP_{A2}. Competitive substrates binding on this enzyme include, but are not limited to: amitriptyline, clomipramine, imipramine, clozapine, olanzapine, haloperidol, ropivacaine, theophylline, zolmitriptan, melatonin, tamoxifen, erlotinib, cyclobenzaprine (a powerful muscle relaxant), estradiol, fluvoxamine, mexiletine, naproxen, ondansetron, phenacetin, paracetamol, propranolol, riluzole, tacrine, tizanidine, verapamil, warfarin (rat poison), zileuton, etc. Expression of CYP_{A2} is induced by hyperinsulinemia, tobacco use, foods including, but not limited to: broccoli, cauliflower, brussels sprouts, etc., nafcillin, omeprazole (a PPI), modafinil (a dopamine agonist), etc. The CYP_{A2} enzyme is inhibited strongly by: ciprofloxacin fluoro-quinolones, fluvoxamine, verapamil, etc.; less strongly inhibited by: essential oils, e.g., chamomile, peppermint, etc., supplements, e.g., dandelion tea, St. John's wort, etc.; and by weak antagonists including, but not limited to: echinacea, cimetidine (a gastric H2 inhibitor), etc.

Presence of competitive ligands, if acute can prolong the bioavailability of caffeine and similar theophyllinic adenosine receptor blockers. However, steady use or presence of such ligands may be expected to induce expression of the enzyme with the result that the ligand is acutely less active; the hyperexpressed (induced) enzyme would be available to more rapidly break down caffeine. Accordingly, a mode of delivery that avoids the liver, for example inhaled or nasally absorbed caffeinic compound can minimize hepatic CYP induction to more reliably exert its beneficial effects. Similar considerations apply with other CYP expression inducers.

Caffeine withdrawal headache is the result of a temporarily increased sensitivity to a natural body chemical and is “nothing to worry about,” according to studies by neuroendocrinologists at the Massachusetts Institute of Technology. The headache is caused by an enhanced response to the natural neurotransmitter, adenosine, one of our chemical messengers that circulate throughout the blood. Adenosine in large, but still considered naturally producible amounts, causes blood vessels to dilate and thus lowers local blood pressure. However, the adenosine levels normally present in the blood was thought to have no effect on blood vessels.

Blood vessel dilation is a known cause of headache, including those diagnosed as migraine headache, and caffeine is often included in headache remedies because it shrinks swelling in nasal passageways to reduce symptoms of sinus headache, sometimes with a bleeding side effect, and helps to constrict, sometimes painfully, dilated blood vessels in the head as well as other bodily locations. M.I.T. headache studies thereby explain how caffeine can be both a cause and a cure of headaches. Recently, researchers at the Johns Hopkins School of Medicine and at the National Institute of Arthritis, Metabolism and Digestive Diseases reported that caffeine affects behavior by countering effects of adenosine in the brain. Adenosine normally inhibits the transmission of messages from one nerve cell to another. Its action resembles that of a tranquilizer, in slowing down brain and other body functions. Caffeine, which is structurally similar to adenosine, blocks this inhibition of nerve message transmission by binding itself to the receptors normally occupied by adenosine. Thus, caffeine acts as a stimulant. Such receptors are cellular beacons to which adenosine would ordinarily attach. Unless linked to a receptor, adenosine is unable to exert physiological effects. Thus, caffeine can decrease sensitivity to adenosine.

Caffeine dosing can span a range with multiple applications of therapy as needed. A per dispense dose of between about 50 mg and 500 mg will generally be effective in delivering caffeine in sufficient quantity for abortive migraine treatment. For those individuals not acclimated to caffeine for cultural, religious or other reasons, a lower dose may suffice. Accordingly, devices may comprise adjustable delivery mechanisms to lessen the amount of caffeine per administration and/or the ratio of caffeine to cannabinolic active compound delivered. Accordingly, a dose per administration may be set, for example, in amounts including, but not limited to about: 10 mg, 20 mg, 25 mg, 35 mg, 50 mg, 60 mg, 75 mg, 90 mg, 100 mg, 120 mg, 125 mg, 140 mg, 150 mg, 175 mg, 200 mg, 225 mg, 250 mg, 275 mg, 300 mg, 325 mg, 350 mg, 400 mg, 425 mg, 450 mg, 475 mg, 500 mg, etc. To put these doses in context, Mountain Dew, a caffeinic beverage that delivers caffeine through the gastrointestinal system contains about 50 mg/serving; AeroLife™ delivers about 100 mg/dose of powdered caffeine (as a dietary supplement) into the mouth. The nasal delivery preferred for delivery in accordance with this invention avoids gastric and circulatory dilution following absorption and thus delivers a significantly higher dose to the site of migraine activity. If preferred by the consumer the device may be reconfigured to administer smaller doses per administration to allow, for example, a balancing between nostrils and/or a sense of greater control by the recipient.

Non-physiologically active ingredients may be present, for example, as preservatives, surfactants, emulsifiers, etc. Benzalkonium chloride is well accepted as a solubilizer and antimicrobial preservative. This, or similar compounds, for example, alternative quaternary ammonia-based surfactant/biocides or mixtures thereof may advantageously be found in the dispensed or dispensable mixture. Benzalkonium chloride is generally used at low concentration (<0.1%) to avoid sensitivity or toxicity concerns.

Polysorbates, e.g., polysorbate 20, 40, 60, 65, 80, etc., and/or PEGs, e.g., PEG 200, 300, 400, etc., comprise acceptably tolerated emulsifying agents whose inclusion in the deliverable of the present invention will be dependent on the degree of emulsification needed based on the active ingredients present in the solution/emulsion.

Water is a well-tolerated and widely applicable solvent. Caffeine, for example when served as coffee of tea is known to be soluble in water in concentrations high enough for desired effects. Essential oils and other nonpolar ingredients may be emulsified or treated with surfactant for delivery in a water matrix. Cellulose derivatives, such as carboxymethylcellulose, hypromellose (hydroxypropylmethyl cellulose) may be added as a viscosity modifier, stabilizer, thickening agent, emulsifier or similar flow or particle size or dryness controlling function, for example, to maintain dispersion, assist in controlling particle size and as a conditioner for maintaining the delivery mechanics. The dispersive, viscosity control properties (as well as its accepted safety profile) are apparent through their use as a moisturizer in artificial tears and anti-redness eyedrops.

In instances where caffeine is being regularly consumed over a period of days, weeks, or longer, adenosine receptors are blocked and unable to react to naturally changing adenosine levels. The body responds by producing more adenosine receptors to rebalance metabolism so that adenosine at normal amounts produced daily in the body can once again exert its normal functions. Chronic caffeine consumption is a known inducer of adenosine receptor expression. When caffeine is stopped abruptly, a larger-than-normal number of receptors become available to bind the normal levels of adenosine. The result, observed in test animals, is a drop in blood pressure in response to only normal circulating levels of adenosine. At least one symptom of addiction to caffeine might be explained by its effect on the action of adenosine.

While pain killers and caffeine can help alleviate migraine symptoms temporarily, there is a societal concern about their side effects. This is one opportunity where CBD (cannabidiol) oil may be a part of a preferred intervention to alleviate the symptoms and minimize such concerns.

Cannabidiol (CBD) is one of the many active compounds found in the cannabis plant. It has grown in popularity as a way to treat several medical conditions naturally.

CBD works by interacting with receptors on cells in both the brain and more distal portions of the nervous system. These receptor molecules have been called cannabinoid receptors (CB₁ and CB₂). Though the relationship between these cells and receptors isn't fully understood, their interaction is thought to affect several systems. For example, CBD may prevent the body from metabolizing anandamide. This is a naturally occurring endocannabinoid compound that is associated with pain perception regulation. Maintaining normal or elevated levels of anandamides in the bloodstream may reduce feelings of pain. CBD is also thought to interact with the immune system to limit inflammation throughout the body, which may also help reduce pain and other secondary immune-system responses, e.g., inflammatory responses. Cannabinoids have also been shown to relax stress hormone induced phenomena.

Cannabinoids and cannabinolic active compounds are a well-known class of substances. For example, at least 20 flavonoid compounds, including, but not limited to: apigenin, quercetin, cannflavin A and cannflavin B, β-sitosterol, vitexin, isovitexin, kaempferol, luteolin and orientin have been identified in the cannabis plant. Other well-known cannabinoids include but are not limited to:
Cannabigerol class: cannabigerolic acid (CBGA) (antibiotic); cannabigerolic acid monomethylether (CBGAM); cannabigerol (CBG) (antibiotic, antifungal, anti-inflammatory, analgesic); cannabigerol monomethylether (CBGM); cannabigerovarinic acid (CBGVA); cannabigerovarin (CBGV).
Cannabichromene class: Cannabichromenic acid (CBCA); cannabichromene (CBC) (antibiotic, antifungal, anti-inflammatory, analgesic); cannabichromevarinic acid (CBCVA); cannabichromevarin (CBCV); Cannabidiolic acid (CBDA) (antibiotic); cannabidiol (CBD) ((antioxidant, anxiolytic, antispasmodic, anti-inflammatory, analgesic); cannabidiol monomethylether (CBDM); cannabidiol C₄ (CBD-C4); cannabidivarinic acid (CBDVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a, 10a-trans-6a,7,8,10a-tetrahydro-6,6,9-trimethyl-3-pentyl-6H-dibenzo[b,d]pyran-1-ol, (Δ⁹ tetrahydrocannabinol, THC) (analgesic, antioxidant, antiemetic, anti-inflammation); Δ⁹-tetrahydrocannabinolic acid-C4 (THCA-C4); Δ⁹-tetrahydrocannabinol-C4 (THC-C4); Δ⁹-tetrahydrocannabivarinic acid (THCVA); Δ⁹-tetrahydrocannabivarinic (THCV); Δ⁷-cis-isotetrahydrocannabivarin; Δ⁹-tetrahydrocannabiorcolic acid (THCA-C1); tetrahydrocannabiorcol (THC-C1).
Δ⁸-tetrahydrocannabinol class: Δ⁸-tetrahydrocannabinolic acid (Δ⁸-TCA); Δ⁸-tetrahydrocannabinol (Δ⁸-THC).
Cannabicyclol class: cannabicyclol (CBL); cannabicyclolicacid (CBLA); cannabicyclovarin (CBLV).
Cannabieson class: cannabiesoic acid A (CBEA-A); cannabiesoic acid B (CBEA-B); cannabieson (CBE).
Cannabinol and cannabinodiol class: cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV).
Cannabitriol class: cannabitriol (CBT); 10-Ethoxy-9-hydroxy-Δ-6a-tetrahydrocannabinol (10-EHDT); 8,9-dihydroxy-delta-6a-tetrahydrocannabinol (8,9-DHDT); cannabitriolvarin (CBTV); ethoxy-cannabitriolvarin (CBTVE).
Miscellaneous class: dehydrocannabifuran (DCBF); cannabifuran (CBF); cannabichromanon (CBCN); cannabicitran (CBT); 10-oxo-Δ-6a-tetrahydrocannabinol (OTHC); Δ⁹-cis-tetrahydrocannabinol (cis-THC); 3,4,5,6-tetrahydro-7-hydroxy-α-α-2-trimethy 1-9-n-propyl-2,6-methano-2H-1-benzoxocin-5-methanol (2H-iso-HHCV); cannabiripsol (CBR); Trihydroxy-Δ⁹-tetrahydrocannabinol (triOH-THC).

Magnolol, a biphenyl neolignan from Magnolia officinalis, acts as a partial agonist for CB₂, while the substance honokiol is less potent, but has full agonistic activity at CB₁ and antagonistic properties at CB₂. Malyngamide B binds both CB₁ and CB₂, with moderate potencies as an agonist anti-inflammatory compound. While many cannabinoids support nitric oxide (NO) production magnolol inhibits NO production with an IC₅₀ ˜6.2 μM. Lower levels of nitric oxide may reduce vasodilation which some attribute as a cause, others as a possible sequella of the migraine process. Some cannabinolic agonists may also exert secondary effects (which some might label as desired side effects).

Vaping CBD oil may be extremely beneficial especially when experiencing a severe migraine at home and you don't have to travel anywhere. The inhalation process delivers the compounds to the bloodstream much quicker than other methods. Currently, there aren't any formal guidelines for proper dosing for a migraine. CBD Oil Review recommends using between 2.5 and 20 milligrams (mg) per day for chronic pain.

Essential oils, such as jasmine and lavender have seen acceptance by a significant number of migraine sufferers. Migraine trigger events or substances as well as migraine relaxants can include scenery, sounds, odors, etc. For example, jasmine produces effects observed in brain scans similar to those after valium administration. While in some instances such may be related to migraine by a Pavlovian association. While such mechanism of action cannot be ruled out, the mechanism is not as important as effective results. Essential oils including, but not limited to: peppermint, sandalwood, rosemary, marjoram, jasmine, geranium, ginger, cinnamon, bergamot, rose, eucalyptus, chamomile, melissa, lemon, basil, thyme, lavender, menthol, etc., have roots dating back over a thousand years in traditional medicine contexts. Lavender and rosemary have been touted as relaxants, e.g., undoing tension triggers bringing on the migraine. Soothing effects may induce a relaxation inducing a sleep or hypnotic state minimizing the nociceptive effects of headache pain. One selling point for the oils is that they prevent or relieve the migraine triggering events and thus are useful for avoiding medications with serious side effects.

The oils can be administered in a variety of ways including, but not limited to: as mouth drops, topical application to hand/wrist/back/belly/forehead, nose spray or drop, nebulizer, mister, bath salt, tea topping, etc. Several of these oils have been associated with one or a variety of known receptors and downstream metabolic pathways. Several have unproven mechanisms of action underlying their noted efficacies. Some may affect nitric oxide and/or endogenous cannabinoid synthesis, transmission, degradation, receptor sensitivity and the like. Some have observed multiple effects relating, for example to cell excitability, blood flow, temperature, sugar uptake, insulin sensitivity, cortisol and/or other hormonal influences, etc. Several flavenoid containing essential oils are alleged to depress nitric oxide (NO) synthesis and/or release. Some also appear to interact with the cannabinoid system antecedent to cannabinoid receptor activity. The arachidonic acid cascade which is used both for endogenous cannabinoid synthesis and prostaglandin synthesis is impacted by several of these flavonoids in vitro. Essential oils may alter membrane fluidity through an unidentified mechanism, possibly by affecting lipid raft predominance and compositions. Membrane transport, intercellular communication, exosome release and other transfer events may be part of several of the oils' mechanisms of action.

So regardless of actual mechanisms of action, treatments including essential oils with or without proven or suspected cannabinoid or other receptor or enzyme agonistic or antagonistic effect, and similarly inhalable or vapable cannabinoids or cannabinolic active substance active for example, on traditional cannabinoid receptors, CB₁ and/or CB₂, but also other cannabinolic active receptors for example receptors including, but not limited to: TRPV₁, TRPV₂, TRPV₃, TRPV₄, TRPA₁, TRPM₅, GPR₁₁₉, GPR₅₅, GPR₁₈, etc. having been individually or as a class shown to be useful are candidates for inclusion in embodiments of this invention.

For example:
Archeologic evidence suggests the use of cannabis for migraine headaches dates back to prehistoric times. Several ancient Greek writings, early documents of Arabic pharmacology, Persian texts from the 10th and 17th centuries and writings from prominent physicians in the Middle Ages share recommendations to use cannabis for headache (or migraine) treatment.
CB₁ and CB₂ are the primary (earliest discovered and often strongest binding) receptors for phyto-cannabinoid compounds such as THC and CBD. Anandamide (AEA) is a prominent endogenous cannabinoid (a compound synthesized in our cells that co-interacts with phyto-cannabinoid receptors like CB₁). Fatty acid amide hydrolase (FAAH) is a major metabolizer of AEA resulting in a decreased availability of AEA for other purposes such as stimulating cannabinoid receptors. An increased level of FAAH activity is observed in many migraine sufferers. At least one study has shown that when used in a preventative context, CBD cut migraine incidences more than 50%. Primary cannabinoid receptor activation thus appears to be one path for decreasing or aborting migraine attacks.
Specifically, with respect to CB₁, a SNP in the CB₁ gene (CNR1) has been correlated with migraine affliction.
Several single nucleotide polymorphs (SNPs) found in the transient receptor potential melastatin 8 ([h]TRPM8) gene have a protective association with migraine. TRPM8 is a nonselective cation channel present at least in subpopulations of small-diameter, cold-sensitive peripheral sensory neurons known to evoke cooling sensations such as menthol and icilin and associated with some Reynaud's patients' early coolness sensations. Both Raynaud's and migraine share vasculature disturbances as part of the disease process. TRPM8 is also associated with pain sensations. Several small molecule antagonists of TRPM8 have shown promise in relieving pain. However, the sites and modes of activities are still under investigation. The prevalence of migraine is significantly increased in Raynaud's patients thus strongly supporting the existence of a shared pathogenetic background or mechanism. Additionally, both migraine and Raynaud's have many of the same triggers. (These may include cigarette smoke; certain medications or chemicals; weather; foods we eat; caffeine; hormones.); both afflict women much more often than men and in women both often co-initiate with menarche; and both seem to be rooted in a hypersensitivity in the nervous system. Care must be taken since treating one condition may exacerbate the other. For example, migraine therapies such as β-blockers, clonidine, and ergots are contra-indicated for Raynaud's because they relieve migraine pain through vasoconstrictive effect and thus can worsen the decreased circulation seen with Raynaud's.
TRPA1, when activated can trigger migraine by stimulating the trigeminal gene-related peptide (CGRP)-dependent pathway; This apparently is an early step in the genesis of pain and other symptoms of migraine attack.
Several CGRP receptor antagonists are effective for abortive treatment of migraine. Antagonists of the TRPV1 ion channel can block the trigeminal activation that leads to the release of CGRP.
Endogenous cannabinoid, anandamide (AEA), a major CB₁ agonist, also agonizes the TRPV1 receptor (at least on trigeminal ganglion neurons) where it stimulates or promote release of CGRP. Antagonizing TRPV₁, e.g., with a blocker or weaker competitive agonizer, is one approach that might be involved in abortive membrane treatment, either as one of the primary active substances or as an adjunctive benefit.

Compounds active against such receptors are utilized for their general calming effects acting through the endogenous cannabinoid receptors systems. Essential oils may work through one or more of these receptors or may exert a referential or Pavlovian type of reflex activity.

Anti-adenosine compounds with theophylline like activity e.g., an inhaler like Aeroshot™ a product recently marketed by Breathable Foods for delivery of caffeine into the lungs or the mouth, brought to the attention of the FDA in 2012 by Senator Schumer. This caffeinic substance and/or other forms of adenosine receptor blocker inhalants or vapors, when inhaled or administered to contact nasal membranes in combination with cannabinolic active compositions represent a distinct improvement in availability of abortive treatments for migraines.

One device of the present invention is preferably configured to comprise a shape such as those used for corticoid administration to nasal passages, i.e., a small projecting delivery portion sized for access to the nasal openings. Larger devices such as masks are also applicable.

Such nasal delivery device comprises an inhaler section, for example, a nasal tube such as used in anesthesia delivery or perhaps a mask that fits over the patient's nasal cavities. The device may comprise a feeder container or mixing bag such as one typically found on airplane oxygen supplies. One or more feeder tubes provides the vapor, aerosol, mist, etc. into the mask or nasal tube optionally though a feeder or mixing container. For variable treatment controls, each tube may deliver a different compound to the patient in the form of a mist, aerosol, vapor gas, etc. The patient inhales the mists provided by the device, and various biofeedback aspects of the patient may be monitored such as body temperature, heart rate, pulse and the like.

The administration of the various compounds may be programmed locally or remotely by a physician or health specialist. The rate of delivery may be controlled as desired, and/or the sequence of delivery of the various compounds may be controlled by the program. The patient can give direct feedback during administration of the medications. The combination of the biofeedback and the human interface allows real-time modification of the medication delivery amounts, sequence etc.

The administration of the various compounds may be programmed locally or remotely by a physician or health specialist. The rate of delivery may be controlled as desired, and/or the sequence of delivery of the various compounds may be controlled by the program. The patient can give direct feedback during administration of the medications. The combination of the biofeedback and the human interface allows real-time modification of the medication delivery amounts, sequence etc.

In several embodiments, the patient can simply control the amounts and sequences of the medication delivery via local controls. These controls may be overridden by limits set in the system and/or by a health care technician to ensure proper delivery, thus providing a control range available to the patient. A sense of control can be an important factor for patient use and compliance.

Addition optional techniques for relaxation therapy may be utilized. Audio and/or visual stimuli, such as virtual reality, may be used to supplement the medications administered to the patient. For example, audio soundtracks for relaxation (e.g. seashore, waves, etc.) may be combined with harmonic vibrational therapy, e.g., about 50 Hz, 60 Hz, 70 Hz, 80 Hz. 90 Hz, etc. frequency. A plurality of frequencies individually variable in amplitude may overly one another. The frequencies may be modulated cycling over a faster or slower rate and or may be pulsated on and off. Adjustment of the audio/visual parameters may be adjusted by the patient as desired.

Prophylactic or preventative treatment for migraines is often tolerated despite serious side effects. Even when used, preventative treatments are not 100% effective resulting in continued suffering in the migraine afflicted person, though possibly with shorter, perhaps less frequent, and often less painful migraine events. Since these prophylaxes are not totally satisfactory treatments to abort a nascent migraine are desired. The present invention teaches a rapidly acting multi-targeted approach for relieving pain and aborting the migraine continuation.

Recognizing that speed is of the essence, the invention, in a preferred embodiment, teaches either buccal or nasal administration for chemical agents needing to be absorbed into the body. The speed of absorption following instillation in the nasal passages and the proximity of the olfactory system to the sight of the migraine render nasal administration, e.g., through an inhaler type device especially preferred as a route of administration.

Treating the migraine etiology, including the pain, which can provide a cycling positive feedback on the event progression, is a multi-targeted approach. A first component targets the adenosine receptor whose hyperactivity (typically when induced by rising adenosine levels) is often an early event in the triggered migraine cascade. A theophylline like substance such as caffeine is used to reduce adenosine receptor binding to adenosine. This should be an acute event as chronic caffeine ingestion has been associated with an increase in adenosine receptor expression and presence in brains of migraine sufferers. An inhalant device for delivering caffeine has been marketed in several countries including the US. Inhaled caffeine will act to abruptly block adenosine induced activities stimulating the migraine event.

A second component of the treatment is use of cannabinolic compounds. These might comprise one of a class of FAAH inhibitors to increase the bioavailability of endogenous cannabinoid compounds, may be endogenous cannabinoid compounds, active metabolites or even precursors to effect greater cannabinoid migraine suppression activity. These might also be exogenous compounds, such as phyto-cannabinolic compounds and/or synthesized compounds to stimulate and maintain cannabinoid receptor activity with the effect of calming the migraine event.

A third possible component is the use of essential oils. These have a history of treating migraines and other afflictions that spans several generations and is found in many cultures. Essential oils are hydrophobic compounds (cell plasma membrane seeking compounds since they prefer the hydrophobic lipid environment). Often these oils have a pleasant and a preferably calming aroma. “Aromatherapy” has used such oils for numerous symptomatic and asymptomatic treatments. In the context of migraines, the efficacy of such treatment is acknowledged without a clear proven mechanism of action. Probably they operate through a multi-mechanistic paradigm. The oils, at least the associated aromas, may exert a sort of Pavlovian conditioning effect providing a reflexive calming. However, many of these oils comprise components with known abilities to interact with one or more of the cannabinoid receptors. Thus, in some embodiments of the invention, the second component may comprise essential oils as a major, perhaps primary cannabinoid for effective treatment.

A caffeinic substance combined with a cannabinolic active component in a device suitable for delivering its contents to the nasal passages is a preferred device for effecting the above described strategies and methods. Such device constitutes another embodiment of this invention.

The drawings and the description below provide a graphic picture of several embodiments of this invention. Embodiments described elsewhere may incorporate one or more features depicted in the drawings.

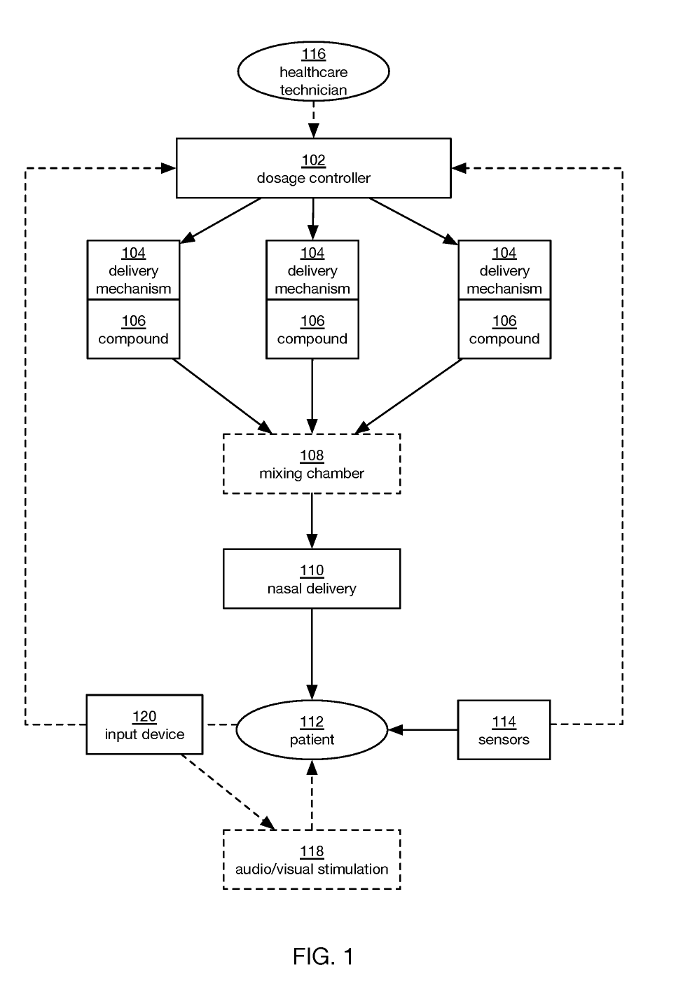

FIG. 1 is a block diagram of a preferred embodiment of the present invention. A dosage controller 102 switching mechanism, which may be some type of computing device including but not limited to a server computer, is connected to one or more delivery mechanisms 104. The dosage controller 102 switching mechanism may switch dosage on/off, may pulse dosage, may select one or more from a plurality of available dosages, may receive manual control feedback from a patient of medical technician, may receive control from a machine that may transmit an analogue and/or digital signal. Each delivery mechanism 104 is associated with a certain compound or composition of compounds 106 that may be delivered in programmable quantities and rates as desired. The compounds or composition of compounds 106 are fed through an optional mixing chamber, which then feeds a nasal delivery device (not shown) as known in the art to the patient 112. The mixing chamber 108 may be optional in embodiments where the compounds may be delivered directly to the nasal delivery device 110 in the specified amounts. The nasal delivery device 110 may be integrated with a mixing chamber 108.

Also shown is optional audio and/or visual stimulation environment 118. This may include speakers or headphones for audio stimulation, as well as a display screen or monitor for visual stimulation. Audio/visual effects such as soothing sounds and images have been shown to help ameliorate the pain of migraine headaches and may be used to supplement the medication delivered through the present invention.

Sensors 114 are biofeedback devices that provide a measurement of various parameters of the patient 112 back to the dosage controller 102. For example, sensors may be employed to monitor the skin temperature of the patient, pulse rate, blood pressure, breathing rate, and the like. This data may be used by the dosage controller to modify and revise the dosages and explained herein.

An input device 120 may be provided to enable the patient to provide some type of manual modification and fine-tune his or her environment. The patient may thusly adapt certain parameters of the audio/visual stimulation environment 118 (e.g. raise or lower the volume of music or other sounds).

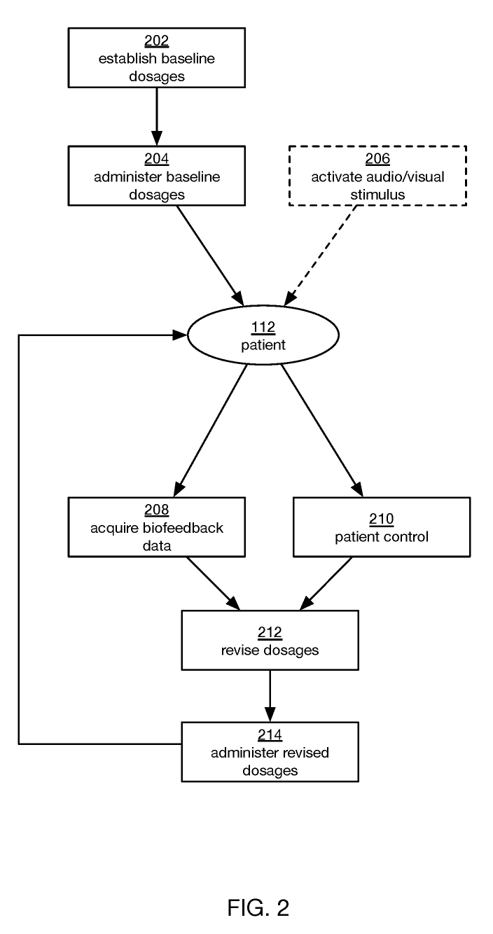

FIG. 2 is a flowchart of the operation of the preferred embodiment of FIG. 1. Initially, the baseline dosages of the various medications that will be administered are established at step 202. This step produces an algorithm that can vary the following parameters of the dosage administration:
Medication selection
Relative amounts of medications delivered
Rate of delivery

Once the baseline dosages algorithm is established, then at step 204 the administration of the baseline dosage is provided. That is, the compounds 106 are delivered via the delivery mechanisms 104 to the mixing chamber 108, wherein the various compounds are mixed together for delivery to the patient. Optionally, at step 206 the audio/visual stimulus is activated.

As the patient 112 receives the dosages via the nasal delivery 110, the sensors 114 will acquire the biofeedback data at step 208. This biofeedback data is then fed to the dosage controller 102 at step 212 so that the dosages may be revised by the dosage controller 102. This may be done in an automated fashion by the dosage controller 102 and/or with input from the healthcare technician 116 and/or patient control 210. Optionally, the patient may enter data that indicates how the treatment is proceeding, which may be used to control the revisions of the dosages. At step 214 the revised dosages are administered, and this process iterates as shown.

Embodiments for treating a migraine event may comprise establishing a baseline dosage of one or more compositions to be administered to a patient via a nasal passage. Such baseline dosage may be established using population or group data, may include input derived from family members, may be based in part on the intended recipient's medical history, etc. For some compounds or compositions, e.g., those with lengthy biological half-lives and few receptor or metabolizing enzyme variants, the established baseline may be an effective introductory dosage for the general population of recipients. Where variants are known, a genetic screen may be instructive in determining effective compounds and dosages thereof.

Baseline dosages having been established sufficient quantities are provided in appropriate reservoirs for controlled delivery. In embodiments with biofeedback affected modulation of dosage, an excess in quantity of at least those compositions that may be modulated upward is suggested. Administering the relevant baseline dosages to the recipient through at least one nasal passage follows. The administering may be effected using any means of connecting reservoirs to at least one nasal passage, for example a nasal tube, a mixing bag, a tent, etc.

As mentioned above, several preferred embodiments comprise acquiring biofeedback data from the patient from patient reported feedback and/or biosensor data that may comprise physiologic data from the recipient. A joystick, button, mouse, slider, microphone, etc., may interface between the recipient and the delivery system. Biosensors may sense parameters relating to the recipient's physiologic status, change in status, rate of change in status and provide data to a dosage controller mechanism to modulate baseline dosage or an adjusted baseline dosage. The biosensor data may include performance data relating to delivering one or more compositions to a nasal opening of the recipient, e.g., compensation for binding to apparatus or system leaks and losses. Data from one or more of these sources may include but are not limited to: temperature, blood flow, oxygenation, pH, air flow volume, air flow rate, humidity, ion conductance, density, sound, luminescence, pressure, etc. These data are applied in modulating or revising the baseline dosages of the one or more medications in accordance with the acquired biofeedback data. The device then introduces the revised dosage(s) of composition(s) for administering to the recipient.

Several embodiments comprise periodic or continuous receiving and processing feedback data from the patient during administration of the mixed dosages. Such data are applied for revising the dosages of the one or more compositions in accordance with the feedback data received from the patient and possible limitation parameters in the system. Dosages may follow rules or guidelines established by a regulatory authority. Revised dosages may hold one or more compound/composition constant, may hold a ratio of a plurality of compositions constant, may be subject to maximum and minimum ratios, may set limits in an algorithm such that increase in one compound/composition may require an increase or decrease in another compound/composition. For example, an increase in one compound may call for a decrease in another when they may share a metabolizing enzyme or when they may have additive or synergistic toxicity. Increasing a compound A may be ill-advised or ineffective absent an increase in a compound B, e.g., when the two are involved in a chemical reaction, when one serves as a cofactor in a pathway of the other, when one may provide a protective shield for the other, etc.

Embodiments of the system and methods may additionally comprise providing an audio, visual and/or olfactory stimulus to the recipient. The system generates or transmits such stimulus to the recipient. The audio and/or visual stimulus may be independent of the composition delivery system or may be incorporated therein. Olfactory stimulus may be introduced through a different nasal passage than used for delivering the compositions or may be delivered using the same mechanism. Olfactory stimulus may be included in at least one of the compositions.

As with composition delivery, embodiments of the invention may comprise receiving and processing feedback data from the patient as a result of the administration of the stimulus. The feedback data may then be used to modulate composition dosages and or olfactory, visual and/or audio stimulus.

Thus, embodiments of the invention include one or more systems for treating a migraine event wherein a dosage controller manages delivery of a plurality of compositions from a reservoir module to a nasal delivery mechanism to connect contents of the reservoir module to at least one nasal passage of a recipient. System embodiments may include at least one biofeedback sensor coupled to the dosage controller such that biofeedback may modulate a baseline dosage relating to delivery of at least one of the plurality of compositions.

The system optionally includes one or more biofeedback sensor that is available for conscious control by a patient and/or a technician. The conscious control may include a shutoff or abort function, may include amplifier circuitry to effect increased delivery of all or a selected composition, may perhaps merely signal mood or degree of pain or the like as data the dosage controller will incorporate with other information or an assigned or selected algorithm and modulate delivery accordingly. Modulation may apply to a single compound or composition, to a ratio between a plurality of compounds/compositions, a positive or negative correlation a pair of compositions.

The dosage controller may be physically connected to the delivery mechanism or may be connected by one or more physical signal including but not limited to: light (including visible light, invisible light, laser, etc.), sound, radio wave (such as wi-fi), etc. to cause delivery of the compositions to at least one nasal passage of the recipient. In several embodiments the system is configured to receive biofeedback from at least one biofeedback sensor and then to adjust delivery of at least one of the compositions in accordance with the received biofeedback.

The system may include one or biofeedback sensor(s) or may be configured to work with independent sensor devices.

### Example A

An inhaler device is instilled with a volatile caffeine substance similar to the product marketed as AEROSHOT™. The inhaler also contains a substance active in the endocannabinoid system. In this first example the phyto-cannabinoid, cannabidiol (CBD), is used for its robust endocannabinolic binding that avoids the induced high” associated with THC. The caffeine/CBD ratio is not critical for most migraine events in most persons. Tolerable ratios generally are in the range of 1 part caffeine per 0.1 parts CBD on a molecular basis. Commonly effective CBD/caffeine ratios may. for example, be about 1:5, 1:8, 1:10, 1:20, 1:25, 1:50, 1:100, etc. In general, on a molecular basis the adenosine binding sites that are the target of the caffeine molecule are quite numerous. Caffeine also will be bound by other proteins including albumin and thus an excess of caffeine over that of CBD provides an immediate migraine abortive effect from the caffeine with sustained caffeine binding supported by the endocannabinoid system under the influence of CBD. Delivery is preferably metered using a metered mechanical or pressurized device.

### Example B

A caffeine containing inhaler device similar to that of Example A is the basis of this example. However, instead of the phyto-cannabinoid CBD, components of essential oils (extracts from various aromatic plants) are incorporated to have endo-cannabinolic effect. Lavender is a well-known essential oil with a history of topical or buccal application to exert a distractive, calming or possibly pain reducing aura. The substance caryophyllene is common to many essential oils and is itself suspected of exerting its influence though endocannabinoid systems. Caryophyllene binds CB₂ and CB₁ in in vitro assays. Accordingly, this Example B comprises an inhalant device configured for releasing caffeine and one or more components of an essential oil based on or derived from lavender. Since caryophyllene is a weaker stimulant of the endocannabinoid systems, and several components of essential oils comprise little or no cannabinolic activity, a higher proportion of essential oil to caffeine will generally be more advantageously effective. Terpenoid compounds including, but not limited to: limonene, myrcene, α-pinene, linalool, caryophyllene oxide, nerolidol, phytol, etc., are listed as examples of other active ingredients to consider as alternatives or co-active components in essential oils that may be featured in one or more embodiments of this invention. Another common component of essential oils is β-hydroxyethylbenzene. It is also added to foods and cosmetics to impart a calming fragrance to the product and as an antimicrobial compound. β-hydroxyethylbenzene is found in many essential oils including, but not limited to: rose hyacinth, orange, geranium, neroli, champaca, carnation, aleppo pine, ylang-ylang, etc.

Understanding that the essential oils will vary in active ingredients and cannabinolic strengths of the components comprised therein the essential oil:caffeine ratio will vary over a broad range. For example, essential oil: caffeine ratio might be configured as about 100,000:1, 50:000:1. 40,000:1, 25,000:1, 20,000:1, 15,000:1, 10,000:1, 5,000:1, 1,000:1, 500:1, etc., by mass. Propellant/solubilizer is selected to solubilize and stabilize the component compounds and provide a delivery matrix. The propellant may be an organic propellant, may be a pumped air propellant, may be a compressed gas propellant, may be an inert propellant such as nitrogen, argon, xenon, etc.

### Example C

An inhaler device is instilled with a volatile caffeine substance similar to the product marketed as AeroLife™ or AEROSHOT™. Aeroshot was designed for inhalation while AeroLife is designed for delivering caffeine to or through the mouth. The essential features are the ability to deliver a metered quantity of contents to a recipient. The inhaler also contains a substance active in the endocannabinoid system. A phyto-cannabinoid or derivative, for example, CBD, tetrahydrocannabivarin, cannabigerol, cannabichromene, etc. The caffeine:cannabinoid ratio is similar to that of Example A. This example comprises the acknowledged cannabinolic compounds from classic cannabinolic sources. In addition, the inhaler device administers essential oils that may have a primary purpose to calm the mind and provide a relaxant aromatic therapy. The essential oils may lack cannabinolic effect, may provide a background or additive cannabinolic effect or may act synergistically. The aerosol delivery device and mechanism may be similar to those used for delivering inhalants of Examples A and B.

### Example D

An alternative dispersal method is used. In place of a misted, sprayed, nebulized, etc., liquid matrix forming droplet particles, at least one active ingredient is dispersed using a gaseous carrier. Similar to the propellant used to disperse the liquid in example B above, the propellant may be an organic propellant, may be a pumped air propellant, may be a compressed gas propellant, may be an inert propellant such as nitrogen, argon, xenon, etc. But rather than propelling liquid of gelled particles, the particle(s) dispersed by this alternate method comprise solid phase particles.

### Example E

This example is provided as a demonstration of the broad applicability of the teachings in this patent application. While delivery through one or more nostril(s) is a preferred delivery method because of the highly controlled delivery achievable, the efficient bioavailability, the speed of delivery and absorption, the common acceptance by a preponderance of candidate patient recipients, the variety of compounds that may be delivered, the migraine abortive treatment is delivered through the eye as droplet(s). This method retains advantages of proximity to the target cells in the cranium, avoids the rare repulsion in some people of any odoriferous substance being nasally instilled, provides alternate formulation possibilities, and does not require intact relatively clear nasal passages. Emulsifiers, calming odors present in the composition, wetting agents, surfactants, etc., may be employed as when a liquid matrix is used as a particulate carrier. For example, glycerine, preferably about 0.2% by volume, may constitute an effective lubricant in such drops. Glycerine or alternative “lubricant” frequently used in eyedrops may be included over a broad range of concentration, for example such lubricant may be sufficiently effective when present in about: 0; 001% by volume, 0.01%, 0.05%, 0.1%, 0.2%, 0.5%, 0.8%, 1%, 2%, 3% 4% 5%, etc. though concentrations in excess of 10% might present sensitivity risk higher than desired. Droplet size of course will not be a major designed feature, but most component constituents can find a place using this delivery method. When present in the tear surface surrounding the eye the more hydrophobic substances will partition to the surface interface between the tear coating and air. Lubrication is desired to ease movement of the eye in its socket and when the eyelid sweeps across the sclera and cornea; antimicrobials preserve the eye drop before disbursement and when in the tear space may afford continued protection; sealers that slow tear evaporation; soothing agents (demulcents) reduce pain and inflammation; and often a vasoconstrictor is incorporated in commercial eye drops to meet cosmetic goals of eliminating redness.

Commonly acceptable soothing agents include but are not limited to: polyethylene glycols (PEG) which are attracted to proteins and glycoproteins associated with mucus membranes provide lubrication and increase viscosity; propylene glycol (PG) which like PEG partitions to the mucus tissue interface has similar properties to PEG in that PG relieves inflammation, increases viscosity and helps retain water; glycerine (aka: glycerin, glycerol), carboxymethylcellulose, polyvinyl alcohol, hydroxymethylcellulose, hydroxypropylcellulose, and hydroxypropylmethylcellulose are some other common soothing agents These make up for insufficient mucins at the lubricated surface. Most common concentrations of these agents in artificial tear products are known in the art from actual marketed products and range from about 0.1% to about 4%.

Available preservatives include but are not limited to: benzalkonium chloride, polyquad, sodium perborate, polyhexamethylene biguanide, chlorobutanol, etc., in concentrations about 0.001% to about 1%.

Since these molecules interact with water they will tend to slow evaporation and reduce sense of dryness. Such additives can therefore be advantageously used to promote comfort and better acceptance of the treatment.

### Example F

Mark, who has a history of migraines, senses an aura which knows usually leads to a debilitating migraine. He puts his tools aside and removes his migraine treatment device from the cabinet, loads his selected cartridges into the device, secures the door, and installs his device over his head and face. Mark first selects delivery of a bolus of caffeine to initiate immediate relief and selects option B for today's abortive intervention. Experience has taught Mark that this option is his best start point for mid-afternoon migraine attacks. The initial bolus compensates for his abstinence from caffeine since breakfast. This option B treatment involves a multi-modal migraine modulating protocol. Mark's unit has been machine programmed by previous use to provide a 65 Hz background sound at 20 dB. A steady delivery of caffeine is provided to both nostrils. After one minute normal in the algorithm to open nasal passages a mist of CBD is infused into the air Mark breathes through his nose. A second tone is added at 78 Hz and 10 dB. The primary ton intensity is gradually increased to 60 dB using a 30 second ramp up. Over a two minute cycle The primary tone is reduced to 20 dB while the second tone is increased to 40 dB. A steady state of tone volume is maintained at each maximum and minimum volume. The two frequencies continue to cycle up and down with each frequency cycling between 20 and 40 dB. in opposite directions. Each cycle minimum to minimum including one plateau occurs over a two minute cycle. At the introduction of the 78 Hz tone an essential oil, jasmine, is periodically spritzed into the nostrils for three seconds during each 45 second interval. Mark is relaxed and feels no inclination to provide a personal override to the machine prepared algorithm. In Mark's cap, sensors provide feedback relating to blood flow to the temple areas of his scalp. Past experience has shown that for Mark as pain increases, scalp blood flow is negatively correlated. For a two hour period the jasmine supplementation continues although the interval between the three second deliveries lengthens to about six minutes. Caffeine delivery tapers to zero over the first forty-five minutes, but the CBD continues for a full ninety minutes that the experientially developed algorithm interfaced with the blood flow data suggests as adequate to prevent furtherance of the migraine. The tones are decreased in amplitude after about seventy-five minutes by continuing the 78 Hz below its 20 dB lower threshold in coordination with the 65 Hz sound reduction at twice the pace so that each reaches silence together.

Mark leans back, sighs, removes the cap, discards the used cartridges and unpacks his tools.

### Example G

Intra-venous injection delivery is used as a dispersal delivery method. While generally this is not the most preferred method for using ingredients as taught in this patent application for arresting a migraine in progress or preventing, for example, an aura from manifesting as a true migraine, intra-venous delivery is possible. Several advantages of the afore-mentioned delivery methods are lost. For example, proximity to the target neuro-cells is not the same. Solid particles would not be compatible with this method. Bioavailability is compromised at least because: a) of the greater distance; b) of circulating proteins and cell walls acting as “sinks” absorbing active ingredients; c) this method is more invasive and more difficult to self-administer; d) calming odors from essential oils are not applicable; and e) patient self-metering of time and amount of treatment is more difficult. Advantages may be to treat patients is the most severe forms of migraine when an i.v. port is already available.

What is claimed is:

1. A device for treating a migraine event, said device comprising:
a) caffeine;
b) a cannabinolic active substance;
c) a carrier liquid; and
d) a delivery device that produces atomized particles.

2. The device of claim 1 wherein the cannabinolic active substance is selected from the group consisting of: CBD, tetrahydrocannabivarin, cannabigerol and cannabichromene.

3. The device of claim 1 wherein the cannabinolic active substance comprises a phyto-cannabinoid or a phyto-cannabinoid derivative.

4. The device of claim 1 wherein the cannabinolic active substance comprises a synthesized cannabinoid.

5. The device of claim 1 wherein the cannabinolic active substance is selected from the group consisting of: apigenin, quercetin, cannflavin A and cannflavin B, β-sitosterol, vitexin, isovitexin, kaempferol, magnolol, luteolin and orientin.

6. The device of claim 1 wherein the cannabinolic active substance comprises a cannabinolic active flavenoid.

7. The device of claim 1 wherein the cannabinolic active substance comprises a cannabinoid of the cannabigerol class.

8. The device of claim 1 wherein the cannabinolic active substance comprises a cannabinoid of the cannabichromene class.

9. The device of claim 1 wherein the cannabinolic active substance comprises a cannabinoid of the Δ⁸-tetrahydrocannabinol class.

10. The device of claim 1 wherein the cannabinolic active substance comprises a cannabinoid of the cannabicyclol class.

11. The device of claim 1 wherein the cannabinolic active substance comprises a cannabinoid of the cannabieson class.

12. The device of claim 1 wherein the cannabinolic active substance comprises a cannabinoid of the cannabinol and cannabinodiol class.

13. The device of claim 1 wherein the cannabinolic active substance comprises a cannabinoid of the cannabitriol class.

14. The device of claim 1 wherein the cannabinolic active substance comprises a cannabinoid of the miscellaneous class.

15. The device of claim 1 wherein the cannabinolic active substance comprises a cannabinolic active composition of essential oil.

16. The device of claim 15 wherein the essential oil comprises jasmine.

17. The device of claim 15 wherein the essential oil comprises lavender.

18. The device of claim 15 wherein the essential oil comprises caryophyllene.

19. The device of claim 1 wherein the cannabinolic active substance comprises a cannabinolic active terpenoid.

20. The device of claim 1 that delivers a metered amount of caffeine per dose.

21. The device of claim 20 wherein said metered amount is included in a range from about 10 mg to about 500 mg per dose.

22. The device of claim 21 wherein said metered amount is included in a range from about 25 mg to about 250 mg per dose.

23. The device of claim 21 wherein said metered amount is included in a range from about 100 mg to about 500 mg per dose.

24. The device of claim 21 wherein said metered amount is included in a range from about 50 mg to about 150 mg per dose.

25. The device of claim 21 wherein said metered amount is included in a range from about 35 mg to about 100 mg per dose.

26. The device of claim 1 wherein a delivered ratio of caffeine to cannabinolic active substance is in a range of about 5 to about 100 on a molecular basis.

27. The device of claim 1 wherein a delivered ratio of caffeine to cannabinolic active substance is in a range of about 0.00001 to about 0.002 by mass.

28. The device of claim 3 further comprising an essential oil.

29. The device of claim 28 wherein the essential oil is selected from the group consisting of: peppermint, sandalwood, rosemary, marjoram, jasmine, geranium, ginger, cinnamon, bergamot, rose, eucalyptus, chamomile, melissa, lemon, basil, thyme, lavender and menthol.

30. The device of claim 1 further comprising:
d) a cap fittable over a person's scalp and at least one of:
e) a sonic device in said cap capable of delivering sound waves into the cranial area;
f) a technological storage device providing stored instruction to said cap;
g) a metering system for delivering said caffeine
h) a metering system for delivering said cannabinolic active substance;
i) a metering system for delivering at least one essential oil;
j) at least one timing system;
k) a connection to a manual control to permit conscious control over the system by the scalp wearer;
l) one or more sensor systems capable of monitoring physiological status; and
m) an interface with one or more of e) through l) to permit monitoring and use of performance or monitoring data to construct or modify a treatment algorithm.

31. A method for relieving or arresting migraine symptoms, said method comprising: providing: a first dose of caffeine and second dose of cannabinolic active substance to a person at risk of or under influence of a migraine event.

32. The method of claim 31 wherein said first dose and said second dose are metered.

33. The method of claim 32 wherein the metered first dose and metered second dose have a ratio in a range about 0.00001 to about 0.002 by mass.

34. The method of claim 32 wherein the metered first dose and metered second dose have a ratio in a range about 5 to about 100 on a molecular basis.

35. The method of claim 31 further comprising providing a third dose, said third dose being of a substance comprising at least one essential oil.

36. A composition for abortive relief of a migraine event, said composition comprising:
a) a carrier matrix capable of dispersal as droplets for delivery through at least one system selected from the group consisting of: the olfactory system, the optical system and the circulatory system;
b) caffeine in a form and quantity effective to bind adenosine receptors in migraine active areas of the brain; and
c) a cannabinolic active substance in a form and quantity effective to interfere with continued migraine activity.

37. The composition of claim 36 wherein the cannabinolic active substance is selected from the group consisting of: CBD, tetrahydrocannabivarin, cannabigerol and cannabichromene.

38. The composition of claim 36 wherein the cannabinolic active substance comprises a phyto-cannabinoid or a phyto-cannabinoid derivative.

39. The composition of claim 36 wherein the cannabinolic active substance comprises a synthesized cannabinoid.

40. The composition of claim 36 wherein the cannabinolic active substance is selected from the group consisting of: apigenin, quercetin, cannflavin A and cannflavin B, β-sitosterol, vitexin, isovitexin, kaempferol, magnolol, luteolin and orientin.

41. The composition of claim 36 wherein the cannabinolic active substance comprises a cannabinolic active flavenoid.

42. The composition of claim 36 wherein said composition is in a form acceptable for use as an eyedrop.

43. The composition of claim 42 further comprising:
d) an optically acceptable demulcent; and
e) an optically acceptable lubricant.

44. The composition of claim 36 wherein said composition is in a form acceptable for use as a nasally active substance.

45. The composition of claim 36 wherein said composition is in a form acceptable for use in intravenous infusion.

46. A method for treating a migraine event comprising:
a) establishing a baseline dosage of one or more medications to be administered to a patient via a nasal passage;
b) providing the baseline dosages of medications to a mixing chamber to form mixed baseline dosages;
c) administering the mixed baseline dosages to the patient through at least one nasal passage;
d) acquiring biofeedback data from the patient;
e) revising the baseline dosages of the one or more medications in accordance with the biofeedback data;
f) providing the revised dosages of medications to the mixing chamber; and
g) administering the mixed revised dosages to the patient.

47. The method of claim 46 further comprising:
h) receiving feedback data from the patient as a result of the administration of the mixed baseline dosages; and
i) revising the baseline dosages of the one or more medications in accordance with the feedback data received from the patient.

48. The method of claim 46 further comprising:
hh) providing an audio/visual stimulus to the patient;
ii) receiving feedback data from the patient as a result of the administration of the audio/visual stimulus; and
jj) revising the baseline dosages of the one or more medications in accordance with the audio/visual feedback data received from the patient.

49. A system for treating a migraine event comprising:
a) a dosage controller;
b) a plurality of medication delivery mechanisms able to receive control instruction from said dosage controller;
c) a plurality of compositions, each comprising at least one compound wherein delivery is modulated in accordance with said control instruction; and
d) a nasal delivery mechanism to connect said dosage controller to at least one nasal passage of a patient.

50. The system of claim 49 further comprising:
e) at least one biofeedback sensor coupled to said dosage controller said biofeedback sensor modulating a baseline dosage relating to delivery of at least one of said plurality of compositions.

51. The system of claim 50 wherein at least one biofeedback sensor is available for conscious control by a patient and/or a technician.

52. The system of claim 50 wherein at least one biofeedback sensor provides physiologic information independent of human conscious control.

53. The system of claim 49 wherein said dosage controller is programmed to establish a baseline dosage of at one of said plurality of dosages and to cause said baseline dosage to be delivered to said nasal delivery mechanism.

54. The system of claim 53 further comprising:
e) interface between at least one biofeedback sensor to a programmable system capable of modulating delivery of at least one of said plurality of compositions.

55. The system of claim 54 used:
to cause delivery of a plurality of compositions to at least one nasal passage of said patient;
to receive biofeedback from at least one biofeedback sensor; and
to adjust delivery of at least one of said plurality of compositions in accordance with said received biofeedback.

56. The system of claim 49 wherein said nasal delivery mechanism comprises a nasal tube.

57. The system of claim 49 wherein said nasal delivery mechanism comprises a mixing chamber.

58. The system of claim 57 wherein said mixing chamber delivers said compositions through a mask.

59. The system of claim 50 further comprising:
an audio/visual stimulus device, under control of a dosage controller programmed:
to cause the audio/visual stimulus device to provide an audio/visual stimulus to the recipient; and
to receive biosensor feedback data from the recipient provided the audio/visual stimulus; and
to revise the baseline dosages of the one or more medications in accordance with the biosensor feedback data received from the recipient.

60. A system for treating a migraine event comprising:
a dosage controller computer;
a plurality of medication delivery mechanisms;
a mixing chamber coupled to the plurality of medication delivery mechanisms;
a nasal delivery mechanism coupled to the mixing chamber; and
one or more biofeedback sensors coupled to the dosage controller computer;
wherein the dosage controller computer is programmed to establish a baseline dosage of one or more medications to be administered to a recipient via a nasal passage;
to cause the mixing chamber to mix the baseline dosages of medications;
to administer the mixed baseline dosages via the nasal delivery mechanism to the recipient;
to receive recipient biofeedback data from the one or more biofeedback sensors;
to revise the baseline dosages of the one or more medications in accordance with the biofeedback data;
to cause the mixing chamber to mix the revised dosages of medications; and
to administer the mixed revised baseline dosages via the nasal delivery mechanism to the recipient.

61. The system of claim 60 further comprising an input device coupled to the dosage controller computer, and wherein the dosage controller computer is further programmed to receive feedback data via the input device from the recipient as a result of the administration of the mixed baseline dosages, and
to revise the baseline dosages of the one or more medications in accordance with the biofeedback data and the feedback data received from the recipient.

62. The system of claim 60 further comprising:
an audio/visual stimulus device, and wherein the dosage controller computer is further programmed to cause the audio/visual stimulus device to provide an audio/visual stimulus to the recipient;
receive audio/visual associated feedback data from the recipient as a result of the audio/visual stimulus; and
revise the baseline dosages of the one or more medications in accordance with the audio/visual associated feedback data received from the recipient.
